(19) United States
(12) Patent Application Publication
(10) Pub. No.: US 2011/0295311 A1
(43) Pub. Date: Dec. 1, 2011

(54) Neck enhancement device

(76) Inventor: Paula Adelman, Bend, OR (US)

(21) Appl. No.: 12/802,092

(22) Filed: May 28, 2010

(51) Int. Cl. A61F 5/08

(52) U.S. Cl. 606/204.35

(57) ABSTRACT

Neck enhancement device with a pair of flexible rectangular adhesive strips, a flexible filament and a filament retaining device. A plurality vertically aligned apertures are located near the end of each strip. The first and second adhesive members are placed next to each other in a mirror-like fashion. The filament is laced through the apertures in the strips. The filament retaining member is slid along the filament and then locked in place. The strips are adhered to the back of a user's neck so that when the filament lace is drawn together, the skin at the rear of the user's neck is drawn together causing the skin at the side and front portion of the user's neck to tighten thereby reducing wrinkles at the side and front portion of the user's neck.

### CROSS REFERENCE TO RELATED APPLICATIONS

Not Applicable

### STATEMENT REGARDING FEDERALLY SPONSORED RESEARCH OR DEVELOPMENT

Not Applicable

### DESCRIPTION OF ATTACHED APPENDIX

Not Applicable

### BACKGROUND OF THE INVENTION

This invention relates generally to the field of cosmetic accessories and more specifically to a neck enhancement device.

As a person ages, the skin of a person tends to loosen and wrinkle. Some people undergo cosmetic surgery to remove unwanted wrinkles in various parts of the body such as the face and neck. However, such drastic cosmetic surgery options are expensive and can be painful. Additionally, the resulting appearance of the individual can look rather un-natural and artificial.

### BRIEF SUMMARY OF THE INVENTION

The primary object of the invention is to provide a portable, inexpensive skin tightening neck enhancement device that can temporarily help a person reduce the visual appearance of wrinkles on a person's neck.

Another object of the invention is to provide a neck enhancement device that is constructed of relatively transparent materials to reduce the visual appearance of the device when in place on a person's neck.

Another object of the invention is to provide a neck enhancement device that can be easily adjusted by the user to control the desired amount of skin tightening.

A further object of the invention is to provide a neck enhancement device that is hypoallergenic.

Yet another object of the invention is to provide a neck enhancement device where a plurality of such devices can be densely packed together for efficient shipping and storage purposes.

Other objects and advantages of the present invention will become apparent from the following descriptions, taken in connection with the accompanying drawings, wherein, by way of illustration and example, an embodiment of the present invention is disclosed.

In accordance with a preferred embodiment of the invention, there is disclosed Neck enhancement device comprising: a first flexible rectangular adhesive strip member, a second flexible rectangular adhesive strip member, a flexible filament member, a filament retention device, said adhesive strip members each folded over at one short end to provide a double thick portion, a plurality vertically aligned apertures located in the region of said double thick portion, said first and second adhesive members placed along side of each other in a mirror-like fashion where said first and second strip apertures are in dose proximity to each other said filament member threaded and laced through said apertures of said first and second adhesive strip members, said filament member threaded through said filament retention device, said filament retention device capable of being slid along said filament member and then locked in place to draw and hold said first adhesive strip member in dose proximity to said second adhesive member, and said first and second adhesive strip members adhered to the back of a user's neck so that when said filament lace is drawn together, said user's skin on said user's neck region between said first and second adhesive strips is drawn together causing the skin at the side and front portion of said user's neck to tighten thereby reducing wrinkles at the side and front portion of said user's neck.

### BRIEF DESCRIPTION OF THE DRAWINGS

FIG. 1 is a front perspective view the neck enhancement device of the invention.

FIG. 2 is a partial perspective view of an adhesive strip of the present invention.

FIG. 3 is a perspective view of the present invention on a user's neck in the loose position.

FIG. 4 is a perspective view of the present invention on the user's neck in the tightened position.

FIG. 5 is a section view of the filament retaining device of the present invention in the clamped position

FIG. 6 is a section view of the filament retaining device of the present invention in the unclamped position.

FIG. 7 is a side view of a person wearing the present invention.

FIG. 8 is a perspective view of a person's collar hiding the presence of the neck enhancement device of the present invention.

FIG. 9 is a plan view of a alternate embodiment of the invention loosened position

FIG. 10 is a plan view of the alternate embodiment of the invention in the tightened position.

FIG. 11 is a top view of the alternate embodiment of the invention.

FIG. 12 is a side view of a second alternate embodiment of the invention.

FIG. 13 is an exploded view of the second alternate embodiment of the invention.

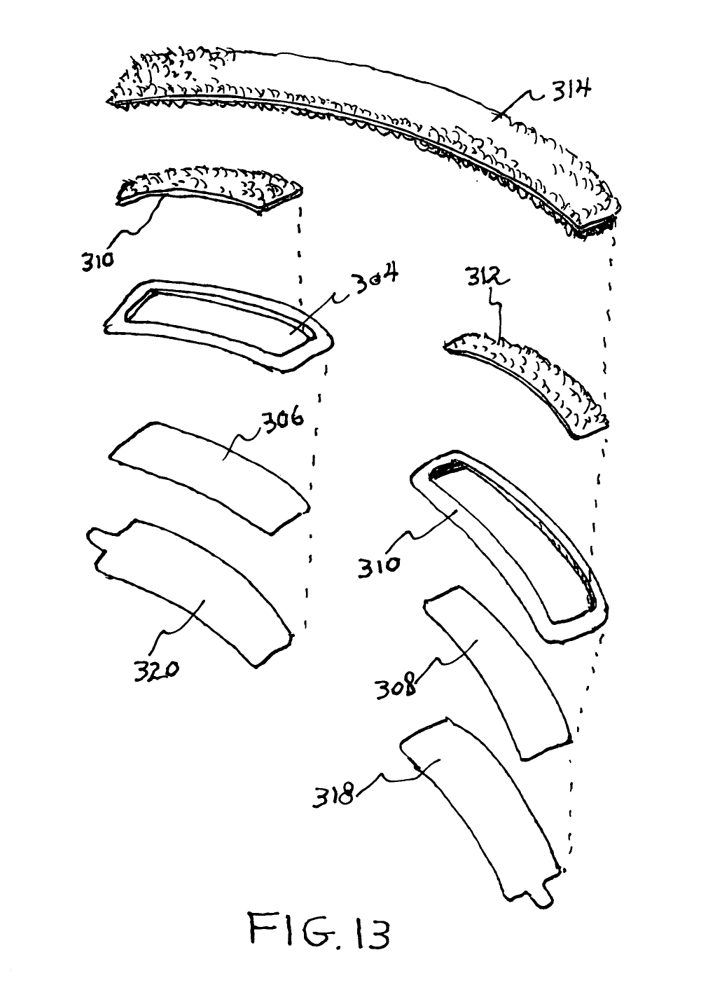

FIG. 14 is a perspective view of the second alternate embodiment of the invention.

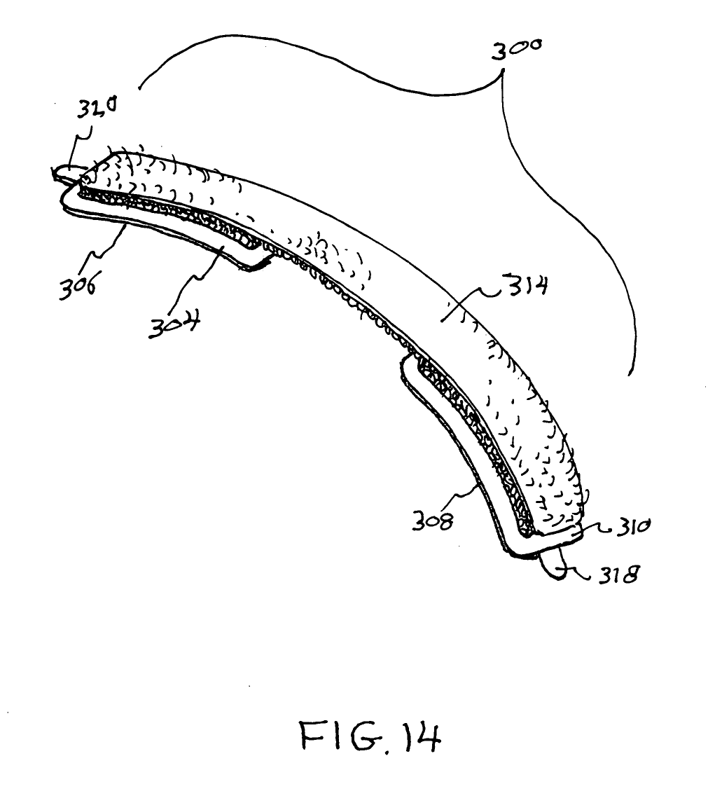

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

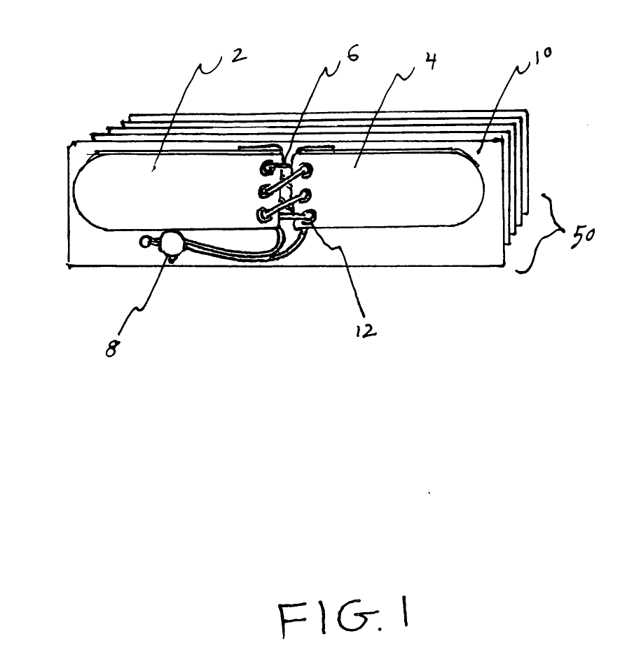

FIG. 1 is a perspective view of the neck enhancement device of the present invention. The device is comprised of a first adhesive strip 2, a second adhesive strip 4, a flexible filament 6 and a filament retaining device 8. The filament 6 is laced through a plurality of apertures 12. The adhesive strips 2, 4 are removably retained on a backing release strip 10. Because of the thin profile of the invention, a plurality of devices 50 can be stored in a compact space for efficient storage and shipment.

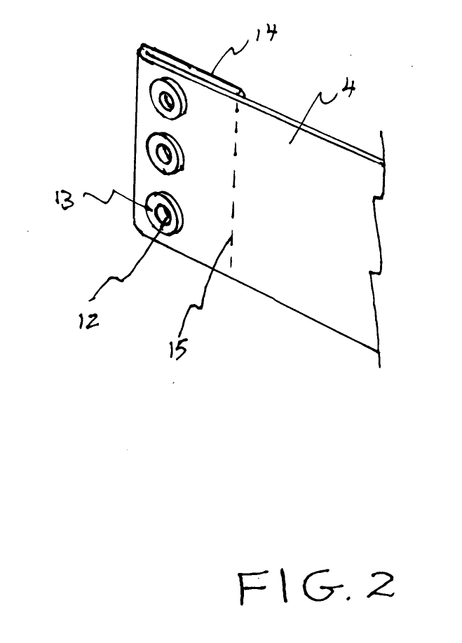

FIG. 2 shows a partial view of one adhesive strip 4. This view shows that a portion of the strip 14 is folded back on itself to create a double thickness. This configuration provides two benefits. First, the strip is reinforced so that the tension of the filament on the apertures 12 is spread over a thicker and stronger region. Second, the folded portion 14 eliminates the adhesive effect at the location of the apertures 12 making it easier to apply the device. Dotted line 15 indicates the folded portion 14. In the preferred embodiments, the apertures 12 are reinforced with transparent plastic eyelets 13.

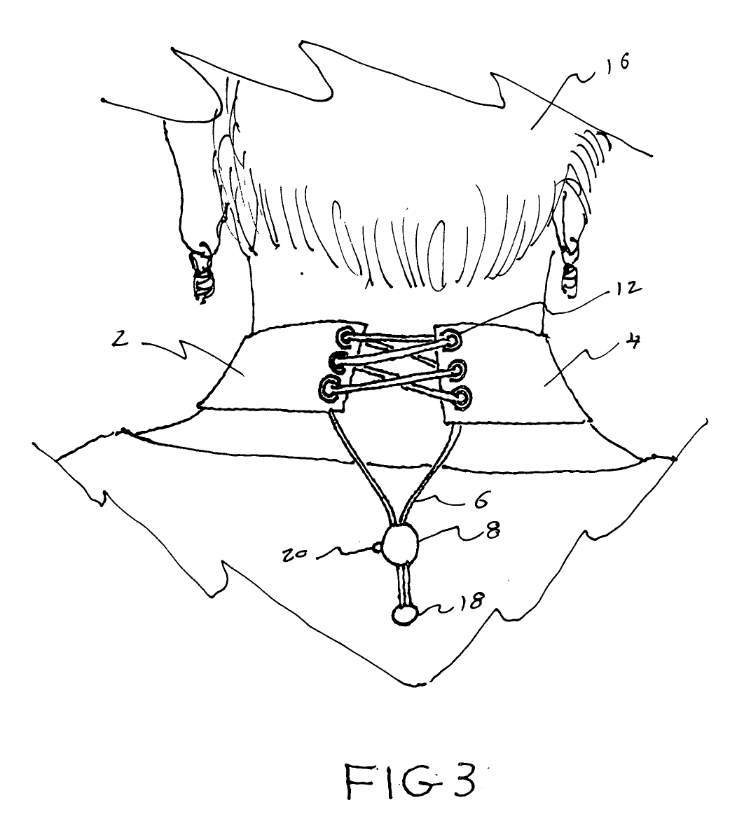

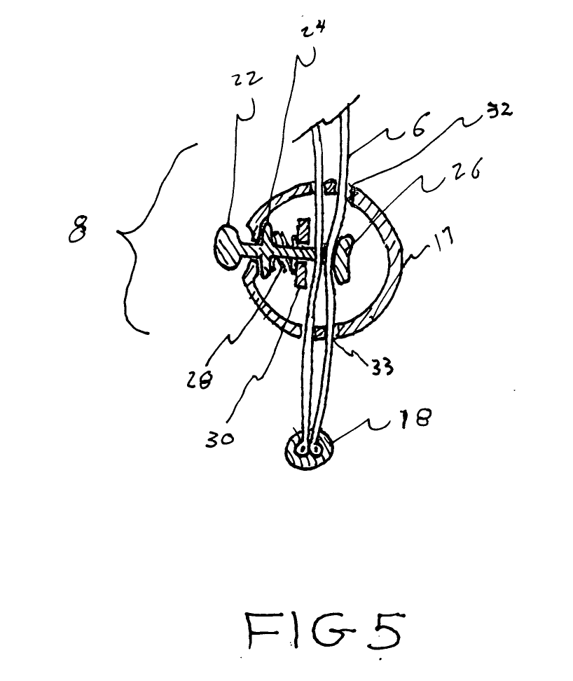

FIG. 3 shows a person 16 wearing the neck enhancement device of the present invention. The user has removed the strips 2, 4 from the backing strip 10 and has placed the first 2 and second 4 adhesive strips on the rear portion of the user's 16 neck. The strips 2, 4 are spread apart by about one and one half inches. The filament 6 ends join together in a parallel fashion and are retained by sliding retaining member 8. The tips of the filament are held together by a tip retaining member 18. To force the two strips 2, 4 together, the user pushes in on push button 20 thereby releasing a retaining post 24, shown in FIG. 5, to allow the retaining member 8 to slide upward. In the preferred embodiment, the filament is made of nylon, similar to twenty pound test fishing line. The filament 6, adhesive strips 2,4, eyelets 13 and the filament retaining member 20 are all made of relatively transparent materials so that the invention does not attract undue attention while being worn. The adhesive strips 2, 4 are made of die cut hypoallergenic polyester.

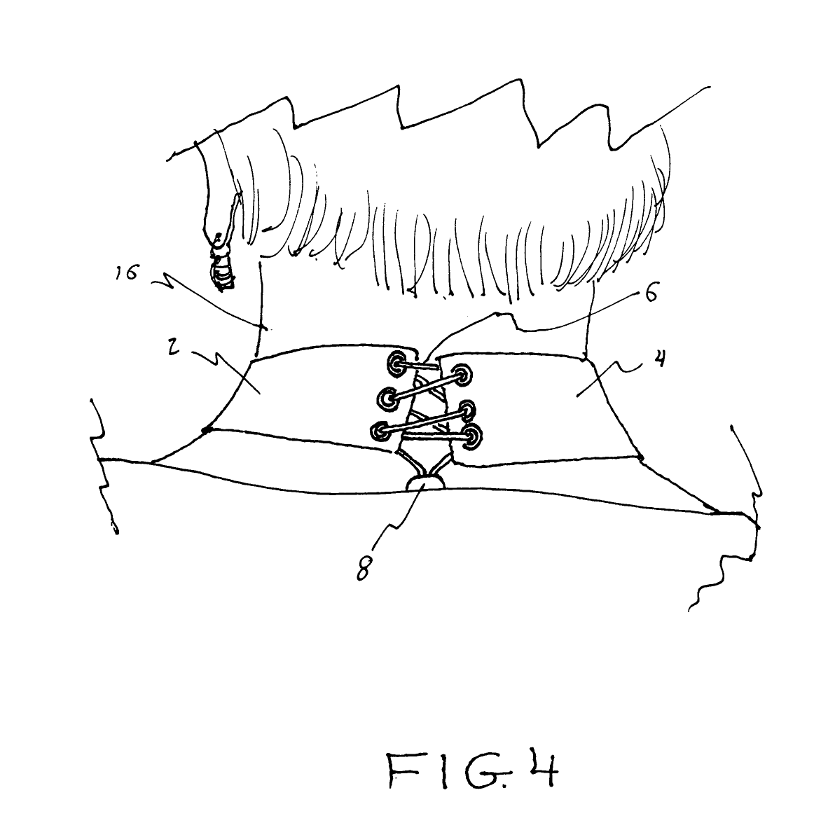

FIG. 4 shows the device in the tightened position. The laced nylon filament 6 has drawn the two adhesive strips 2, 4 toward each other and in the process, caused the skin located between the two strips 2, 4 to be squeezed together and causing the skin at the side and front of the user's neck to be pulled back and be tightened thereby reducing the appearance of wrinkles on the user's neck. The user 16 can select and control the degree of skin tightness pulling on or loosening filament member 6 and then by sliding and locking the retaining member 20 as high as it will go toward the adhesive strips 2, 4. Filament retaining device 8 has been partially hidden by the user's shirt.

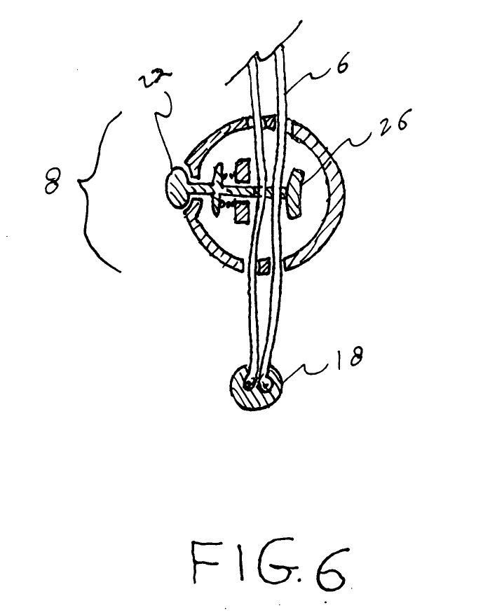

FIG. 5 shows a front section view of the retaining device 8. The two filament ends of filament 6 extend through the housing 17 through apertures 32, 33. A post 24 terminates in a push button 22 at one end and in a filament squeezing tab 26 at the opposite end. A compression spring 28 holds the tab 26 firmly against the filament 6 unless it is forcibly pushed away when a user pushes on push button 22 as shown in FIG. 6. Spring restraining tab 30 is fixedly mounted to the floor of housing 17. The shoulder on post 24 prevents the post 24 from leaving the housing 17. Filament tip retainer fob 16 holds the filament ends together and prevents the housing 17 from being slid completely off of the filament 6.

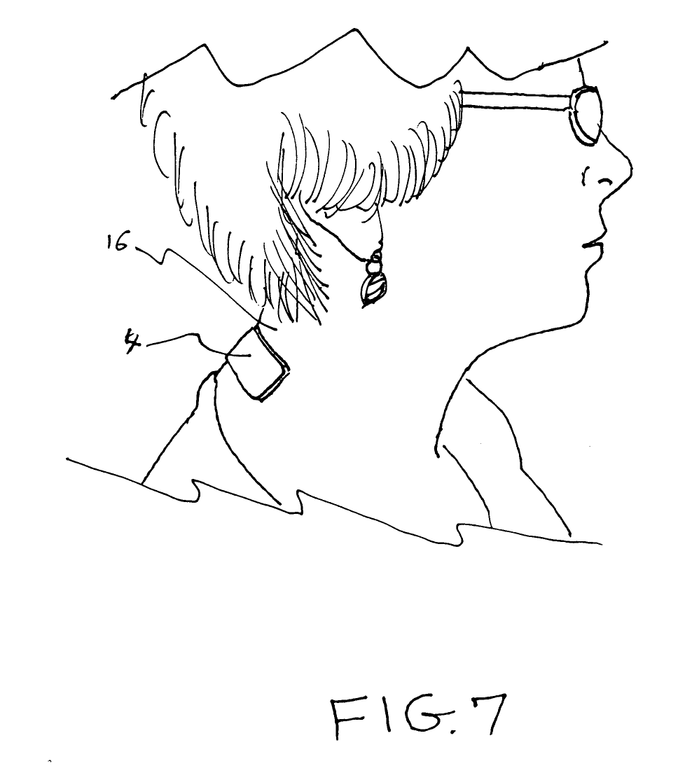

FIG. 7 shows a perspective side view of a person 16 wearing the present invention. Adhesive strip 4 is in view, however, since the strip 4 is made of relatively transparent material, it does not call attention to itself.

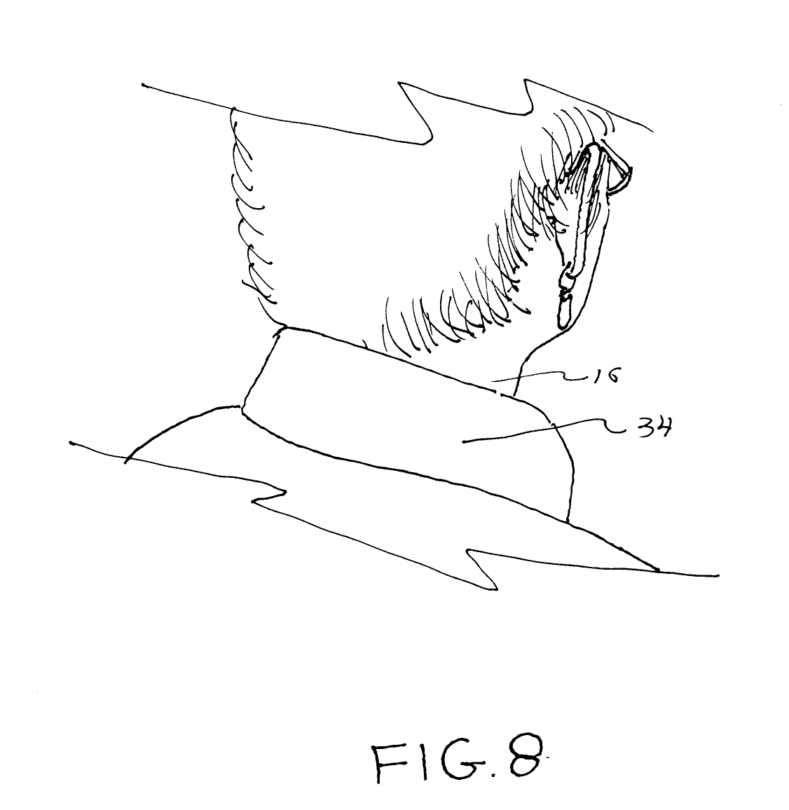

FIG. 8 shows a person 16 wearing a shirt with a collar thereby completely hiding the present invention from view. In this way, the wearer can look years younger in the neck region without anyone realizing that the smooth looking neck skin is a result of a temporary, portable device of the present invention worn by the user.

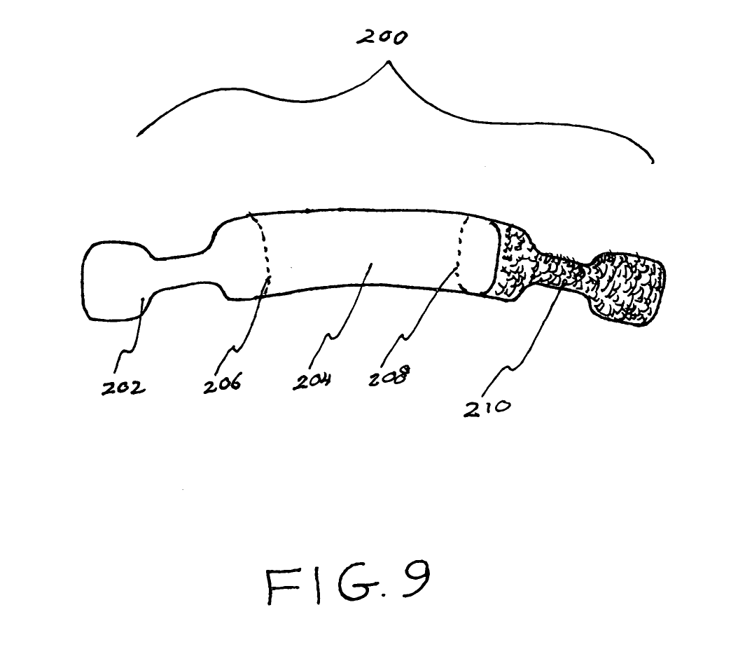

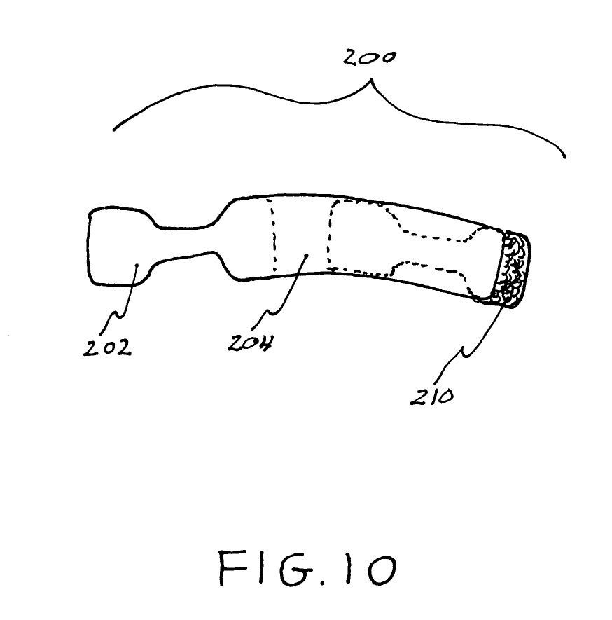

FIG. 9 shows an alternate embodiment of the invention 200. In this embodiment an adhesive strip 202 is fixedly attached to a loop type fastening strip 204. Dotted line 206 shows the overlap between strip 202 and fastener strip 204. An adhesive strip on the opposite side 210 has hook type fastening material fixed to it The hook strip 210 is removably overlapped and attached to the loop strip 204 as shown by dotted line 208. To use this alternate embodiment 200, the user removes protective backing strips on adhesive members 202 and to 210 and places the assembly 210 on the back of his or her neck. The user then releases the overlapped portion of loop strip 204 and pulls the strip 204 toward strip 210 thereby pulling the user's neck skin, and then re-attaching loop strip 204 to hook strip 210 as shown in FIG. 10. The contoured shape of the assembly 200 allows it to more accurately follow the contour of a user's neck when the assembly 200 is installed on a neck.

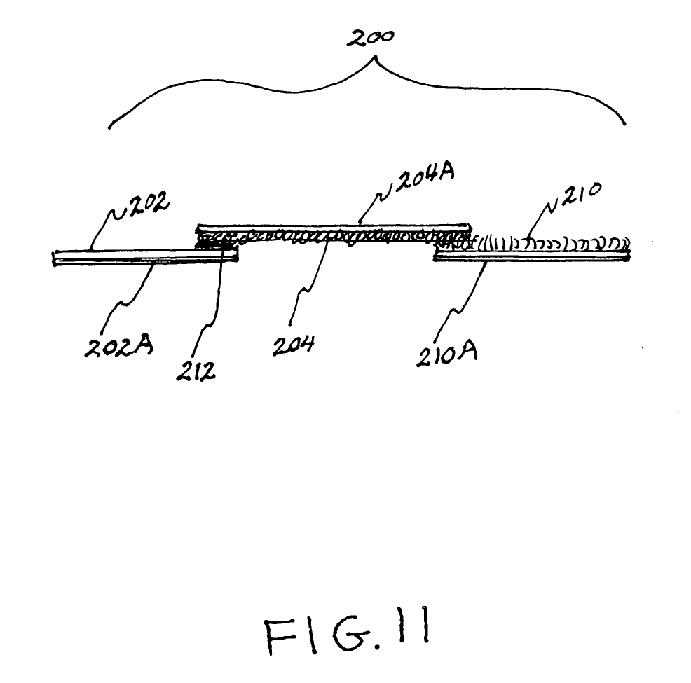

FIG. 11 shows a top view of the alternate embodiment 200. Adhesive strip 202 includes a protective release strip 202A that is removed just prior attachment to the user's neck. Hook strip 210 also includes a protective release strip 210A that is removed just prior to attachment to the user's neck. Loop strip 204 also includes an adhesive side that is protected by a protective release strip 204A. This adhesive portion provides an option for the user to removably attach a decorative neckerchief to the strip 204 which covers the assembly 200 and hides it from view from the public. The neckerchief hangs loosely around the user's neck thereby not hiding the front portion of the user's neck. The final result is a pulling and tightening of the user's neck skin so that the skin at the front portion of the user's neck looks wrinkle free, while the assembly 200 is conceited from the public by a neckerchief. The strip 202 is fixedly attached to strip 204 at area 212 by standard attachment means such as hot melt glue.

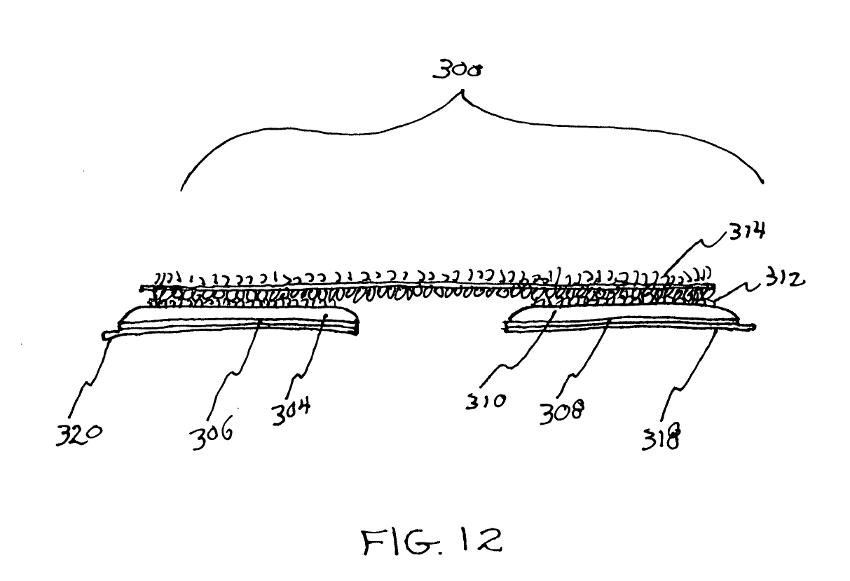

FIG. 12 shows a side view of a second alternate embodiment of the invention 300. In this embodiment a pair of flexible molded holding members 304, 308 each fixedly retain a strip of hook type fastening material 310, 312. A long strip of double sided hook and loop material 314, having the loop side facing down and the hook side facing up, can adjustably connect the two strips of hook material 310, 312. The hook side of strip 314 can removably engage a neck scarf which can cover the entire assembly 300 while it is in place on the user's neck. Double sided tape strip 306, 308 are located on the underside of holder pieces 304, 314. A protective release strips 318, 320 are removed just before application to the user's neck. The device 300 is placed onto the back of the user's neck and pressed in place via double sided tape 306, 308. The user then lifts up on one end of strip 314 off of loop fastener 310 and pulls it toward the loop fastener 310 until the skin between the two loop fasteners 310, 312 is squeezed together as described in the other embodiments of the invention. In the present embodiment 300, the user can remove and replace the double sided tape strips 306, 308 while re-using the rest of the assembly 300

Other types of removable attachment means may be employed in the alternate embodiment, other than hook and loop type fasteners, such as snaps, buttons, or wire form latches such as those found on bra closures.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

What is claimed is:

1. Neck enhancement device comprising:
a first flexible rectangular adhesive strip member;
a second flexible rectangular adhesive strip member;
a flexible filament member;
a filament retention device;
said adhesive strip members each folded over at one short end to provide a double thick portion;
a plurality vertically aligned apertures located in the region of said double thick portion;
said first and second adhesive members placed along side of each other in a mirror-like fashion where said first and second strip apertures are in close proximity to each other
said filament member threaded and laced through said apertures of said first and second adhesive strip members;
said filament member threaded through said filament retention device;
said filament retention device capable of being slid along said filament member and then locked in place to draw and hold said first adhesive strip member in close proximity to said second adhesive member; and
said first and second adhesive strip members adhered to the back of a user's neck so that when said filament lace is drawn together, said user's skin on said user's neck region between said first and second adhesive strips is drawn together causing the skin at the side and front portion of said user's neck to tighten thereby reducing wrinkles at the side and front portion of said user's neck.

2. Neck enhancement device as claimed in claim 1 wherein said apertures are reinforced with eyelets.

3. Neck enhancement device as claimed in claim 1 wherein said adhesive strips are made of hypoallergenic polyester material.

4. Neck enhancement device as claimed in claim 1 wherein said filament member ends are paired together in close proximity to each other and held together by a filament retaining fob.

5. Neck enhancement device as claimed in claim 1 wherein said adhesive strips and said eyelets and said filament are constructed of relativity transparent materials.

6. Neck enhancement device as claimed in claim 1 wherein said adhesive strips are removably adhered to a backing release panel.

7. Neck enhancement device as claimed in claim 1 wherein a plurality of said adhesive strips and attached said backing panels can be stacked together in a compact manner for efficient storage and shipping.

8. Neck enhancement device as claimed in claim 1 wherein said filament is made of twenty pound test nylon.

9. Neck enhancement device as claimed in claim 1 wherein said filament retention device is comprised of two rigid halves forming a housing and a spring biased retaining post;
said post exiting the side wall of said housing and terminating in a push button;
said housing having filament side wall apertures allowing said filament pair to enter and exit said housing and
said filament retention device capable of being slid up or down on said filament pair when said push button is pressed, and lock in place when said push button is released.

10. Neck enhancement device as claimed in claim 1 wherein said user can control the amount of neck tightening by selecting the amount of pull on said filament member and then locking in the selected amount of tightness by the use of said locking filament retaining member.

11. Alternate embodiment of the device as claimed in claim 1 wherein hook and loop fastening strips act to provide the skin tightening means.

12. Alternate embodiment of the device as claimed in claim 11 that includes an outwardly facing adhesive strip that allows a user to removably attach a neckerchief to said hook and loop strip assembly.

13. A second alternated embodiment of the device as claimed in claim 11 wherein said hook fastening strips are each fixedly retained in flexible holding members and where said holding members include removable and replaceable double sided adhesive strips mounted to the underside of said holding members that attach the said holding members to said user's neck.
